(19) United States
(12) Patent Application Publication
(10) Pub. No.: US 2009/0287007 A1
(43) Pub. Date: Nov. 19, 2009

(54) PARTIALLY-HYDROGENATED, FULLY-EPOXIDIZED VEGETABLE OIL DERIVATIVE

(75) Inventors: Timothy Walter Abraham, Minnetonka, MN (US); Charles Michael Tanger, Minneapolis, MN (US)

Correspondence Address: CARGILL, INCORPORATED, P.O. Box 5624, MINNEAPOLIS, MN, 55440-5624, US

(73) Assignee: CARGILL, INCORPORATED, Wayzata, MN (US)

(21) Appl. No.: 12/465,303

(22) Filed: May 13, 2009

Related U.S. Application Data
(60) Provisional application No. 61/127,383, filed on May 13, 2008.

(51) Int. Cl. C07D 303/42; C07D 301/03

(52) U.S. Cl. 549/524; 549/561

(57) ABSTRACT

Disclosed is a method for making a partially hydrogenated, fully epoxidized vegetable oil derivative. The method includes fully epoxidizing a partially hydrogenated vegetable oil having an iodine value of 70 to 100 g I₂/100 grams oil to obtain a partially hydrogenated, fully-epoxidized vegetable oil derivative exhibiting a iodine value less than 4 g I₂/100 gram, an acid number less than 1 mg KOH/gram, an EOC from 4.0 to 5.7% and a Gardner Color value of 1 or less.

### CROSS REFERENCE TO RELATED APPLICATION

This application claims the benefit of U.S. Provisional Patent Application Ser. No. 61/127,383 filed May 13, 2008 entitled PARTIALLY-HYDROGENATED, FULLY EPOXIDIZED VEGETABLE OIL DERIVATIVE, which is hereby incorporated by reference in its entirety.

### FIELD

This invention relates to partially-hydrogenated, fully-epoxidized vegetable oil derivatives. And, in some particular aspects, partially-hydrogenated, fully-epoxidized soybean oil derivatives.

### BACKGROUND

Polyols are generally produced from petroleum-derived feedstocks. Polyols have been used in a variety of applications, including coatings, adhesives, sealants, elastomers, resins and foams. Polyurethane foams are a particularly large end-use market where polyols are used.

Recently non-petroleum based polyols have become available. These non-petroleum based polyols can be produced from vegetable oils.

Some examples of non-petroleum based polyols include those described in U.S. Pat. Nos. 6,107,433, 6,433,121, 6,573,354, and 6,686,435 as well as PCT Publications WO 2006/012344 A1 and WO 2006/116456 A1.

### SUMMARY

Partially-hydrogenated, fully-epoxidized vegetable oil derivatives are described that are suitable for the manufacture of bio-based polyols that surprisingly result in white foams, which are resistant to yellowing, even in the absence of ultraviolet light stabilizers. The fully-epoxidized vegetable oil derivatives and polyols made therefrom have very low levels of unreacted double bonds. Low density flexible polyurethane foams incorporating polyols made from the vegetable oil derivatives of the invention have little or no tendency to cross-link when high compression forces are applied. Therefore, such polyurethane foams are very soft and resilient compared to foams made from polyols containing large numbers of unreacted carbon-carbon double bonds.

The partially-hydrogenated, fully-epoxidized vegetable oil derivatives are low in color, having a Gardner Value of 1 or less and have low odor characteristics, as measured by total volatiles based on hexanal, nonanal, and decanal of 25 ppm or less.

In some preferred aspects, the partially-hydrogenated, fully-epoxidized vegetable oil derivative is made from a partially hydrogenated vegetable oil having at least 50% monounsaturated fatty acid groups, more preferably at least 65% monounsaturated fatty acid groups, further more preferably at least 70% monounsaturated fatty acid groups; and less than 40% saturated fatty acid groups, more preferably less than 25% saturated fatty acid groups, further more preferably less than 20% saturated fatty acid groups, and in some instances less than 15% saturated fatty acid groups, for example less than 10% saturated fatty acid groups. This high level of monounsaturated fatty acid groups and the low level of saturated fatty acid derivatives will result in a more even distribution of epoxy groups in the vegetable oil derivative for a starting oil having a given iodine value. It is believed the more even distribution of epoxy groups will lead to a more homogeneous polyol, in particular, a polyol having a narrower molecular weight distribution than a polyol made from an epoxidized vegetable oil derivative, which was made from a partially hydrogenated vegetable oil having higher levels of saturated fatty acid groups and lower levels of monounsaturated fatty acid groups.

In a particularly preferred aspect of the invention the partially-hydrogenated. fully-epoxidized vegetable oil derivative is made from a partially hydrogenated vegetable oil having a iodine value from 70 to 100 grams I₂/100 grams oil, more preferably from 85 to 95 grams I₂/100 grams oil, which was made by hydrogenating a refined, bleached, and deodorized vegetable oil having a starting iodine value of from 120 to 140 grams I₂/100 grams oil and more commonly from 120 to 135 grams I₂/100 grams oil. While not intending to be bound by any theory, it is believed the hydrogenation of the vegetable oil removes/modifies undesirable chemical species within the vegetable oil that tend to cause yellowing in the foams manufactured using polyols made from the vegetable oil. And, the low levels of unsaturation further reduce the potential for color bodies to be formed by the interaction of the unsaturated carbon-carbon double bonds with ambient light. The use of partially hydrogenated vegetable oil having the described iodine value facilitates the manufacture of a polyol having a hydroxyl number between 40 and 80 mg KOH/gram polyol and a relatively high molecular weight, with a very low iodine value (i.e. low residual carbon-carbon double bonds achieved through fully-epoxidizing the oil), and low residual epoxy functionality (i.e. low EOC values). Surprisingly, low density flexible polyurethane foams made from such a polyol are very resistant to yellowing caused by ambient light exposure.

### DETAILED DESCRIPTION

### Terms and Definitions

As used herein “polyol” refers to a molecule having an average of greater than 1.0 hydroxyl groups per molecule. A polyol may also include functionality other than hydroxyl groups.

“Fully-epoxidized” or “fully-epoxidizing” refers to treating a vegetable oil to modify its chemical structure to replace the carbon-carbon double bonds of the oil with epoxy groups. The resulting molecule is referred to as a fully-epoxidized vegetable oil derivative. In order for a vegetable oil derivative to be fully-epoxidized, it is not necessary to react all the carbon-carbon double bounds within the oil. However, the iodine value of the vegetable oil derivative should be reduced to a level of 4 grams I₂/100 gram vegetable oil derivative or less.

The term “partially hydrogenated vegetable oil” refers to a vegetable oil that has been treated with hydrogen or a source of hydrogen to convert a portion of the carbon-carbon double bounds into carbon-carbon single (saturated) bonds. During the hydrogenation process, the iodine value of the vegetable oil reduces.

“EOC” refers to epoxy oxygen content, which is the weight percent of epoxy oxygen for the material of interest. EOC is determined according to the procedure of ASTM D1652 (manual method—modified to use 50 ml of 5.3% solution of tetraethylammonium bromide in acetic acid). EOC is reported as percent (%).

“Iodine Value” (IV) is defined as the number of grams of iodine that will react with 100 grams of material being measured. Iodine value is a measure of the unsaturation (carbon-carbon double bonds and carbon-carbon triple bonds) present in a vegetable oil, epoxidized vegetable oil derivative, or polyol. Iodine Value is reported in units of grams iodine (I₂) per 100 grams material and is determined using the procedure of AOCS Cd Id-92.

“Hydroxyl number” (OH#) is a measure of the hydroxyl (—OH) groups present in a polyol. It is reported in units of mg KOH/gram polyol and is measured according to the procedure of ASTM E1899-02.

“Number average molecular weight” (Mn) is determined according to the procedure delineated in the Examples and is reported in units of Daltons.

“Acid Value” (AV) is a measure of the residual hydronium groups present in a compound and is reported in units of mg KOH/gram material. The acid number is measured according to the method of AOCS Cd 3d-63.

“Viscosity” for purposes of this invention is reported in units of pascal-seconds (Pa.s) and is measured at 25° C. according to the procedure of ASTM D2196.

“Gardner Color Value” is a visual measure of the color of a vegetable oil, epoxidized vegetable oil derivative, and/or polyol. It is determined according to the procedure of ASTM D1544, “Standard Test Method for Color of Transparent Liquids (Gardner Color Scale)”. The Gardner Color scale ranges from colors of water-white to dark brown defined by a series of standards ranging from colorless to dark brown, against which the sample of interest is compared. Values range from 0 for the lightest to 18 for the darkest, For the purposes of the invention, the Gardner Color Value is measured on a sample of material at a temperature of from 35 to 40° C.

“IFD” refers to the “indentation force deflection value” which is a measure of the load bearing quality of a foam. IFD is typically expressed in Newtons per 323 square centimeter at a given percentage deflection of the foam and measured in accordance with ASTM D3574.

“Support Factor” is Firmness at 65% IFD/Firmness at 25% IFD.

“Fn” is the number average hydroxyl functionality expressed in average number of hydroxyl groups per polyol molecule. Fn is calculated using the equation:

Fn=(OH#/56)*(Mn/1000)

“Peroxide Value” is a measure of the peroxide chemical species (hydroperoxides, peroxides, etc) present in a material. It is measured according to the method of AOCS Cd 8b-90 (2003), and is reported in units of milliequivalent peroxide/1000 grams (meq/1000 grams).

“Total volatiles based on hexanal, decanal, and nonanal” are measured according to the following: a 20 ml headspace vial containing 0.5 grams of sample and 3 microliters of an internal standard (50 microgram/mL ethylbenzene in pentane) is equilibrated at 50 C for 20 minutes. A SPME fiber (divinylbenzene/Carboxan/Polydimethyl siloxane) is inserted into the headspace for 20 minutes. The SPME fiber is desorbed for 1 minute at 240 C in the injection port of a gas chromatograph, and eluted through an HP-5 capillary column (30 m×0.25 mm×0.25 micrometer) programmed from 40 C to 100 C at 10 C/min., then from 100 C to 250 C at 20 C/min. with a final hold of 5.5 minutes at 250 C. The concentration of the aldehydes are calculated based on the ratios of the aldehyde peaks to internal standard compared to a calibration curve for a set of ethylbenzene/aldehyde standards.

### Partially Hydrogenated Vegetable Oil:

Prior to hydrogenation, the vegetable oil typically has an iodine value of at least 120 grams I₂/100 grams oil. The vegetable oil typically is hydrogenated sufficiently to obtain a final iodine value of from 70 to 100 grams I₂/100 grams oil. Preferably, partially hydrogenated vegetable oils having a iodine value of from 85 to 95 grams I₂/100 grams oil are utilized in the invention. A partially hydrogenated vegetable oil having a iodine value between 85 and 95 grams I₂/100 grams oil will lead to a partially hydrogenated, fully-epoxidized vegetable oil derivative that exhibits better fiowability at room temperature (25° C.) than partially hydrogenated vegetable oils having lower iodine values. Polyols made from partially hydrogenated, fully-epoxidized vegetable oils having iodine values less than 70 grams I₂/100 grams oil tend to be waxy solids at room temperature and therefore are difficult to handle and utilize. For polyols made from partially hydrogenated vegetable oils having iodine values from 70 to 80 grams I₂/100 grams oil, preferably the polyols are heated before reacting with a polyisocyanate and/or the process used for the reaction is heated.

In a particularly prepared aspect where oils having a starting iodine value of from 120-135 g I₂/100 grams oil are used, preferably, the iodine value of the partially hydrogenated vegetable oil is reduced by at least 18%, more preferably at least 20%, and further more preferably by at least 22% from the initial iodine value of the non-hydrogenated vegetable oil.

The vegetable oil utilized preferably is refined, bleached and deodorized (“RBD”) using methods known to one of ordinary skill in the art. Preferably, the vegetable oil is refined, bleached and deodorized prior to being hydrogenated. Examples of vegetable oils suitable for use in the invention include: soybean oil, sunflower oil, corn oil, canola oil, and safflower oil.

Partial hydrogenation can be conducted according to any known method for hydrogenating double bond-containing compounds such as vegetable oils. Catalysts for hydrogenation are known and can be homogeneous or heterogeneous (e.g., present in a different phase, typically the solid phase, than the substrate). One useful hydrogenation catalyst is nickel. Other useful hydrogenation catalysts include copper, palladium, platinum, molybdenum, iron, ruthenium, osmium, rhodium, iridium, zinc or cobalt. Combinations of catalysts can also be used. Bimetallic catalysts can be used, for example, palladium-copper, palladium-lead, nickel-chromite.

In some aspects, the catalysts can be impregnated on solid supports. Some useful supports include carbon, silica, alumina, magnesia, titania, and zirconia, for example. Illustrative support embodiments include, for example, palladium, platinum, rhodium or ruthenium on carbon or alumina support; nickel on magnesia, alumina or zirconia support; palladium on barium sulfate (BaSO₄) support; or copper on silica support.

Commercial examples of supported nickel hydrogenation catalysts include those available under the trade designations “NYSOFACT,” “NYSOSEL,” AND “NI 5248 D” (from Englehard Corporation, Iselin, N.J.). Additional supported nickel hydrogenation catalysts include those commercially available under the trade designations “PRICAT 9910,” “PRICAT 9920,” “PRICAT 9908” and “PRICAT 9936” (from Johnson Matthey Catalysts, Ward Hill, Mass.).

The catalysts may be deployed in a fixed bed. The catalyst also may be finely dispersed within the vegetable oil being hydrogenated. A system where a supported catalyst is finely dispersed within the vegetable oil to be reacted is often referred to as a slurry phase reaction.

The metal catalysts can be utilized with promoters that may or may not be other metals. Illustrative metal catalysts with promoter include, for example, nickel with sulfur or copper as promoter: copper with chromium or zinc as promoter; zinc with chromium as promoter; or palladium on carbon with silver or bismuth as promoter.

Partial hydrogenation can be carried out in a batch, continuous or semi-continuous process. In a representative batch process, a vacuum is pulled on the headspace of a stirred reaction vessel and the reaction vessel is charged with the vegetable oil to be hydrogenated (for example, RBD soybean oil). The material is then heated to a desired temperature, typically in the range of about 50° C. to about 350° C., for example, about 100° C. to about 300° C., or about 150° C. to about 250° C. The desired temperature can vary, for example, with hydrogen gas pressure. Typically, a higher gas pressure will require a lower temperature. In a separate container, the hydrogenation catalyst is weighed into a mixing vessel and is slurried in a small amount of the vegetable oil to be hydrogenated (for example, RBD soybean oil). When the vegetable oil reaches the desired temperature (typically a temperature below a target hydrogenation temperature), the slurry of hydrogenation catalyst is added to the reaction vessel. Hydrogen is then pumped into the reaction vessel to achieve a desired pressure of H₂ gas. Typically, the H₂ gas pressure ranges from about 15 psig to about 3000 psig, for example, about 15 psig to about 90 psig. As the gas pressure increases, more specialized high-pressure processing equipment can be required. Under these conditions the hydrogenation reaction begins and the temperature is allowed to increase to the desired hydrogenation temperature (for example, about 120° C. to about 200° C.), where it is maintained by cooling the reaction mass, for example, with cooling coils. When the desired degree of hydrogenation is reached, the reaction mass is cooled to the desired filtration temperature.

In preferred aspects, hydrogenation is conducted in a manner to promote selectivity toward monounsaturated fatty acid groups, i.e., fatty acid groups containing a single carbon-carbon double bond. Selectivity is understood here as the tendency of the hydrogenation process to hydrogenate polyunsaturated fatty acid groups over monounsaturated fatty acid groups. This form of selectivity is often called preferential selectivity, or selective hydrogenation.

The level of selectivity of hydrogenation can be influenced by the nature of the catalyst, the reaction conditions, and the presence of impurities. Generally speaking, catalysts having a high selectivity in one fat or oil also have a high selectivity in other fats or oils. As used herein, “selective hydrogenation” refers to hydrogenation conditions (e.g., selection of catalyst, reaction conditions such as temperature, rate of heating and/or cooling, catalyst concentration, hydrogen availability, and the like) that are chosen to promote hydrogenation of polyunsaturated compounds to monounsaturated compounds. Using soybean oil as an example, the selectivity of the hydrogenation process is determined by examining the content of the various C18 fatty acids groups within the vegetable oil and their ratios. Hydrogenation on a macro scale can be regarded as a stepwise process:

k₃ k₂ k₁

C18:3→C18:2→C18:1→C18:0

The following selectivity ratios (SR) can be defined: SRI=k₃/k₂; SRII=k₂/k₁. Characteristics of the starting oil and the hydrogenated product are utilized to determine the selectivity ratio (SR) for each fatty acid group. This is typically done with the assistance of gas-liquid chromatography. For example, the vegetable oil may be saponified to yield free fatty acids (FFA) by reacting with NaOH/MeOH. The FFAs are then methylated into fatty acid methyl esters (FAMEs) using BF₃/MeOH as the acid catalyst and MeOH as the derivatization reagent. The resulting FAMEs are then separated using a gas-liquid chromatograph and are detected with a flame ionization detector (GC/FID). An internal standard is used to determine the weight percent of each of the fatty esters (i.e., saturated, monounsaturated, and polyunsaturated). The rate constants can be calculated by either the use of a computer or graph. as is known. The above-described method is also utilized to determine the fatty acid groups (i.e. those groups derived from monounsaturated fatty acids, polyunsaturated fatty acids, and saturated fatty acids present in the partially hydrogenated vegetable oil).

In addition to the selectivity ratios, the following individual reaction rate constants can be described within the hydrogenation reaction: k₃ (linolenic to linoleic and other diunsaturated fatty acids), k₂ (linoleic and other diunsaturated fatty acids to oleic and other monounsaturated fatty acids), and k₁ (oleic and other monounsaturated fatty acids to stearic). In some preferred aspects, the inventive method involves hydrogenation under conditions sufficient to provide a selectivity or preference for k₂ and/or k₃ (i.e., k₂ and/or k₃ are greater than k₁). In these aspects, then, hydrogenation is conducted to reduce levels of polyunsaturated compounds within the starting material, while minimizing generation of saturated compounds.

In one illustrative embodiment, selective hydrogenation can promote hydrogenation of polyunsaturated fatty acid acyl groups toward monounsaturated fatty acid acyl groups (having one carbon-carbon double bond), for example, tri- or diunsaturated fatty acid acyl groups to monounsaturated groups. In some preferred embodiments, the invention involves selective hydrogenation of a vegetable oil (such as soybean oil) to a hydrogenation product having a minimum of 50% monounsaturated fatty acid groups, more preferably a minimum of 65% monounsaturated fatty acid groups, and further more preferably a minimum of 70% monounsaturated fatty acid groups, and a maximum of 40% saturated fatty acid groups, more preferably a maximum of 25% saturated fatty acid groups, and further more preferably a maximum of 20% saturated fatly acid groups.

After partial hydrogenation, the hydrogenation catalyst can be removed from the partially hydrogenated vegetable oil using known techniques, for example, by filtration. In some embodiments, the hydrogenation catalyst is removed using a plate and frame filter such as those commercially available from Sparkle Filters, Inc., Conroe, Tex. In some embodiments, the filtration is performed with the assistance of pressure or a vacuum. In order to improve filtering performance, a filter aid can optionally be used. A filter aid can be added to the hydrogenated product directly or it can be applied to the filter. Representative examples of filtering aids include diatomaceous earth, silica, alumina and carbon. Other filtering techniques and filtering aids can also be employed to remove the used hydrogenation catalyst. For example, in other embodiments, the hydrogenation catalyst is removed by using centrifugation followed by decantation of the product.

### Epoxidation:

The partially hydrogenated vegetable oil described above is typically epoxidized using a peroxyacid under conditions that fully epoxidize the carbon-carbon double bonds present within the vegetable oil. For purposes of the invention, in order to fully epoxidize the carbon-carbon bonds within the oil, all the double bonds do not have to be epoxidized, but enough should be epoxidized to reduce the iodine value of the resulting fully-epoxidized vegetable oil derivative to 4 grams KOH/100 gram vegetable oil derivative or less. Typically, another acid (in addition to peroxyacid) will be used during the epoxidation reaction.

Examples of peroxyacid that may be used include peroxyformic acid, peroxyacetic acid, trifluoroperoxyacetic acid, benzyloxyperoxyformic acid, 3,5-dinitroperoxybenzoic acid, m-chloroperoxybenzoic acid, and combinations thereof. Preferably, peroxyformic acid or peroxyacetic acid will be utilized. The peroxy acid may be added directly to the reaction, or may be formed in-situ by reacting a hydroperoxide compound with a acid such as formic acid, benzoic acid, acetic acid or fatty acids such as oleic acid. Examples of hydroperoxides that may be used include hydrogen peroxide, tert-butylhydroperoxide, triphenysilylhydroperoxide, cumylhyroperoxide, and combinations thereof. Most preferably hydrogen peroxide will be used. Preferably, the amount of acid used to form the peroxyacid is from about 0.25 to about 1.0 moles of acid per mole of carbon-carbon double bonds in the vegetable oil, and more preferably from about 0.45 to about 0.55 moles of acid per mole of carbon-carbon double bonds in the vegetable oil. Preferably, the amount of hydroperoxide used to form the peroxy acid is 0.5 to 1.5 moles of hydroperoxide per mole of double bonds in the vegetable oil, and more preferably 0.8 to 1.2 moles of hydroperoxide per mole of double bonds in the vegetable oil.

The final EOC of the partially-hydrogenated, fully epoxidized vegetable oil derivative is from 4.0% to 5.7%, preferably from 4.3% to 5.7%. and more preferably from 4.5% to 5.41%. This relatively low EOC level will assist in the manufacture of a polyol having a high molecular weight, but still having a relatively low value for EOC.

As discussed above, an additional acid component is typically also included in epoxidation reaction mixture. Examples of suitable additional acid components include sulfuric acid, para-toluenesulfonic acid, hydrofluoric acid, trifluoroacetic acid, fluoroboric acid, Lewis acids, acidic clays, or acidic ion exchange resins.

Optionally, a solvent may be added to the epoxidation reaction. Suitable solvents include chemically inert solvents such as aprotic solvents. For example, these solvents do not include a nucleophile, and are non-reactive with acids. Hydrophobic solvents, such as aromatic and aliphatic hydrocarbons, are especially desirable. Examples of suitable solvents include benzene, toluene, xylene, hexane, pentane, heptane, and chlorinated solvents, such as carbon tetrachloride. Preferably, toluene will be used if a solvent is used in the reaction mixture. Solvents are useful in that they may be used to control the speed of the reaction and to reduce the number of undesirable side reactions. The solvent also reduces the viscosity of the reaction mixture and the viscosity of the mixture containing the product. This reduced viscosity aids the processing of the partially hydrogenated fully-epoxidized vegetable oil derivative.

The reaction product may be neutralized to reduce any remaining acidic components in the reaction product. Suitable neutralizing agents include weak bases, metal bicarbonates, and ion-exchange resins. Examples of neutralizing agents that may be used include ammonia, calcium carbonate, sodium bicarbonate, magnesium carbonate, amines, and ion exchange resins. An example of a suitable weakly-basic ion-exchange resin is Lewatit MP-64 ion-exchange resin (available from Bayer Corporation). The acid value of the partially hydrogenated fully-epoxidized vegetable oil derivative is less than 1 mg KOH/gram vegetable oil derivative.

The partially hydrogenated, fully-epoxidized vegetable oil derivative has a Gardner Color value of 1 or below, and in preferred aspects contains 25 ppm or less total volatiles based on hexanal, nonanal and decanal. In order to achieve this low volatile level, the partially hydrogenated, fully-epoxidized vegetable oil derivative preferably is deodorized. Preferably, the deodorizing step occurs after the product has been washed to remove impurities, such as acids. During the deodorizing step, the vegetable oil derivative is heated to a temperature of at least 170° C., preferably at least 180° C., more preferably at least 190° C. Volatiles such as hexanal, decanal, and nonanal are removed from the vegetable oil derivative, during and/or after the heating step. The vegetable oil derivative is typically heated to a sufficient temperature and for a sufficient length of time to reduce the peroxide value of the vegetable oil derivative to less than 10, preferably less than 8, more preferably less than 6, and in some circumstances less than 4 meq)/1000 grams. Typically, the vegetable oil derivative will be heated to a temperature from 170° C. to 210° C. for a period of time sufficient to reduce the peroxide value to the above-described levels. Preferably, the vegetable oil derivative should not be heated above a temperature of 220° C., to reduce any degradation of the vegetable oil derivative. Reducing the peroxide values to these low levels will also significantly reduce any odors present in polyols made from the vegetable oil derivative, particularly the levels of total volatiles based on hexanal, decanal, and nonanal.

### Polyols and Polyurethane Foams:

The partially-hydrogenated, fully epoxidized vegetable oil derivative typically can be reacted with a ring opener (nucleophile) to form a polyol that can be utilized in the manufacture of low density, flexible polyurethane foams. The polyol has relatively low values of EOC, typically less than 2.8 percent by weight. The typical ring opener utilized includes alcohol, water and other compounds having one or more nucleophilic groups. The most preferred ring openers are C1 to C4 monohydric alcohols.

The polyols can be reacted with an isocyanate compound, typically in the presence of a catalyst and blowing agents as known to those of ordinary skill in the art to form low density (5 to 97 Kg/m³) and/or very low density (8 to 24 Kg/m³), flexible polyurethane foams having excellent resistance to yellowing as measured by a low yellowness index (YI) after being exposed to ambient light. Due to its low values for EOC as described above, the polyol of the invention is particularly useful for very low density polyurethane foam (i.e. those foams having densities from 8 to 24 Kg/m³. For very low density foams, it has been found that the foams may be susceptible to scorching in the presence of phosphorus and chlorine based additives, and that maintaining the EOC level of the polyol below the values described above, will minimize any such scorching.

The flexible foams are a flexible cellular product. In a particularly preferred aspect, the flexible foam will not rupture when a specimen 200 by 25 by 25 mm is bent around a 25-mm diameter mandrel at a uniform rate of 1 lap in 5 seconds at a temperature between 18 and 29° C., according to the procedure of ASTM D3574.

The low density, flexible, yellowing resistant polyurethane foams may be made utilizing any of the typical manufacturing methods known to one of ordinary skill in the art. For example, slabstock and molded polyurethane foam manufacturing methods may be utilized.

Examples of conventional slabstock foaming equipment/processes include, for example, commercial box-foamers, high or low pressure continuous foam machines, crowned block processes, rectangular block processes (e.g. Draka, Petzetakis, Hennecke, Planiblock, EconoFoam, and Maxfoam processes), and verti-foam processes.

### EXAMPLES

The following examples exemplify methods for making a partially hydrogenated, fully epoxidized vegetable oil derivative. The examples also show methods for making polyols from partially hydrogenated, fully epoxidized vegetable oil derivatives and flexible polyurethane foams made from such polyols.

### Materials:

Refined, bleached soybean oil (RBSBO): A refined, bleached soybean oil having an iodine value of 125-135 grams I₂/100 grams oil, available from Cargill, Incorporated.

PHSBO-60: A partially-hydrogenated soybean oil having an iodine value of about 60.6 grams I₂/100 grams oil made by hydrogenating a refined, bleached soybean oil having an initial iodine value of 125 to 135 grams I₂/100 grams oil using a procedure similar to the hydrogenation procedure described below.

PHSBO-75: A partially-hydrogenated soybean oil having an iodine value of about 74.6 grams I₂/100 grams oil made by hydrogenating a refined, bleached soybean oil having an initial iodine value of 125 to 135 grams I₂/100 grams oil using a procedure similar to the hydrogenation procedure described below.

PHSBO-80: A partially-hydrogenated soybean oil having an iodine value of about 79.2 grams I₂/100 grams oil made by hydrogenating a refined, bleached soybean oil having an initial iodine value of 125 to 135 grams I₂/100 grams oil using a procedure similar to the hydrogenation procedure described below.

PHSBO-83: A partially-hydrogenated soybean oil having an iodine value of about 83.1 grams I₂/100 grams oil made by hydrogenating a refined, bleached soybean oil having an initial iodine value of 125 to 135 grams I₂/100 grams oil using a procedure similar to the hydrogenation procedure described below.

PHSBO-90: A partially-hydrogenated soybean oil having an iodine value of about 90.1 grams I₂/100 grams oil made by hydrogenating a refined, bleached soybean oil having an initial iodine value of 125 to 135 grams I₂/100 grams oil using a procedure similar to the hydrogenation procedure described below.

PHSBO-100: A partially-hydrogenated soybean oil having an iodine value of about 100 grams I₂/100 grams oil made by hydrogenating a refined, bleached soybean oil having an initial iodine value of 125 to 135 grams I₂/100 grams oil using a procedure similar to the hydrogenation procedure described below.

PHSBO-105: A partially-hydrogenated soybean oil having an iodine value of about 105 grams I₂/100 grams oil made by hydrogenating a refined, bleached soybean oil having an initial iodine value of 125 to 135 grams I₂/100 grams oil using a procedure similar to the hydrogenation procedure described below.

PHSBO-110: A partially-hydrogenated soybean oil having an iodine value of about 110 grams I₂/100 grams oil made by hydrogenating a refined, bleached soybean oil having an initial iodine value of 125 to 135 grams I₂/100 grams oil using a procedure similar to the hydrogenation procedure described below.

Ni Catalyst: A hydrogenation catalyst in tablet form containing 20-25% by weight Nickel and 75-80%) by weight tristearin available from Johnson Mathey.

Dowex C-211: An acidic cationic exchange resin available from The Dow Chemical Company.

Ring-Opening Catalyst: an aqueous solution of 48% by weight hydrofluoroboric acid (HBF₄/H₂O), available from EMD Sciences.

Polyol Arcol F-3022: is a 3,000 MW polyether polyol with an OH# of 56 KOH/grams and a nominal Fn of 3.

TDI: Lupranate T80 is 80%-20% mixture of the 2,4 and 2,6 isomers of toluene diisocyanate available from (BASF).

Niax L-5770 (silicone surfactant): is a polyether modified siloxane. available from Momentive Performance Materials.

Dabco BL-11 (amine catalyst): is a 70% dilution of bis(dimethylaminoethylether) in Dipropylene Glycol available from Air Products.

Kosmos K-29 (Stannous octoate): KOSMOS® 29 is the stannous salt of ethylhexanoic acid. It is also known under the name stannous octoate. Kosmos 29 is available from Evonik Industries.

Hydrogen peroxide solution: An aqueous solution of 30% by weight H₂O₂.

Acetic Acid (99.7%): Glacial acetic acid available from EMD Sciences.

Ring Opener: Methanol (99.8%) available from EMD Sciences.

Toluene ACS 99.5% available from Alfa Aesar.

“Number average molecular weight (Mn) and weight average molecular weight (Mw)” are measured by Gel Permeation Chromatography (GPC) using a Waters High Performance Liquid Chromatography (HPLC) Pump Model #1525, a Waters 717 plus Autosampler, and a Waters 2410 Refractive Index detector (all available from Waters Corporation). The samples are eluted from PLgel columns (highly crosslinked porous polystyrene/divinylbenzene matrix) from Varian Polymer Laboratories connected in series, in the following order, two PLgel, 5 micrometer, 300×7.5 mm, 50 Angstrom (Å) columns, followed by one PLgel, 5 micrometer, 300×7.5 mm, 500 Å column. The columns are maintained at 50° C. A 10 microliter volume of a 2% solution of the sample in tetrahydrofuran (THF) is injected into the columns and eluted with THF at 1 ml/minute.

Mn and Mw are calculated using “Breeze” software available from Waters Corporation. The software calculates Mn and Mw using a second-order polynomial calibration curve based on the following standards: the following materials are used as number average molecular weight (Mn) standards: Arcol LHT-240 (Mn=700 Daltons), Soybean oil (Mn=874 Daltons), Epoxidized soybean oil (Mn=940 Daltons), Acclaim 2200 (Mn=2008 Daltons), Multranol 3400 (Mn=3000 Daltons) and Acclaim 8200 (Mn=7685 Daltons).

### 1. Hydrogenation of Refined, Bleached Soybean Oil:

Approximately 900 grams of RBSBO and 0.9 grams of Ni Catalyst are charged into a 2 liter stainless steel reactor manufactured by Parr Instrument Co., (Moline, Ill.) equipped with a thermocouple, an external heating mantle, temperature (heat only) controller, and an internal stirring mechanism. The reaction vessel is closed and air is purged from the oil by sparging nitrogen through the oil for approximately six minutes. After sparging, heat is applied to the vessel to raise the temperature of the oil to approximately 140° C., and the internal stirring mechanism is activated to stir the oil at approximately 500 revolutions per minute (RPM). After the temperature stabilizes at 140° C. hydrogen gas at a pressure of 50 psig is applied to the reaction vessel. A gas pressure regulator maintains a constant hydrogen gas pressure of approximately 50 psig on the reaction vessel throughout the hydrogenation reaction. The supply of hydrogen gas to the reaction vessel is ceased after sufficient reaction time has lapsed, and the vessel is purged with nitrogen for approximately ten minutes. The partially-hydrogenated soybean oil product is removed from the reaction vessel and filtered to remove the Ni Catalyst from the partially-hydrogenated soybean oil. Partially-hydrogenated soybean oils having desired iodine values are obtained by varying the length of time of the hydrogenation reaction.

### 2. Full Epoxidation of the Partially-Hydrogenated Soybean Oil Derivatives:

### EXAMPLES 1-1 THROUGH 1-8

700 grams of Partially-hydrogenated soybean oil (PHSBO), together with Dowex C-211, Acetic Acid and toluene according to the parts by weight listed in Table 1, are charged to a 2-Liter 3-neck round-bottom flask equipped with a thermocouple, heating mantle, temperature (heating only) controller, mechanical stirrer, and addition funnel. The reaction mixture is heated with stirring to 70° C. The heat is turned off and approximately one-fifth (⅕) of the Hydrogen Peroxide solution indicated in Table 1 is added to the vessel through the addition funnel at a rate to maintain the temperature recorded by thermocouple below 80° C. The remaining hydrogen peroxide solution is added incrementally over approximately 1 to 1.5 hours while maintaining the thermocouple temperature below 80° C. After approximately six hours, the reaction is complete. The stirring is stopped and the reaction product is allowed to cool. Once the reaction product is cooled, the Dowex C-211 resin settles to the bottom of the flask and is separated from the liquid by decanting off the liquid. The decanted liquid separates into an aqueous phase and an organic rich phase. The organic rich phase containing the fully epoxidized vegetable oil derivative is washed approximately five times with water until the aqueous phase has a pH of 7. Toluene and residual water are removed from the washed organic phase by heating the washed organic phase to 90° C. under reduced pressure (final pressure of approximately 2-3 torr) for 1 to 2 hours. The properties of the epoxidized vegetable oil derivative are listed in Table 2. All the epoxidized soybean oil derivatives (“PHFESBO”) are a pale yellow in color and have a Gardner Color value of 1 or less when measured at 35 to 40° C. As can be seen from Table 2, epoxidized soybean oil derivatives made from soybean oils having an initial iodine value of less than 70 gram I₂/100 grams oil (Example 1-1) are waxy solids at room temperature and have values for EOC which are too low to enable the ready manufacture of high molecular weight polyols having the desired hydroxyl number. Further, the polyols made from these epoxidized soybean oil derivatives will be solids having relatively high melting points, which will make them difficult to handle in most polyurethane reaction processes. The fully epoxidized soybean oil derivatives made from partially hydrogenated vegetable oil having iodine values above 100 g I₂/100 gram oil have undesirably high values for EOC. The high values for EOC of Examples 1-7 and 1-8 will be difficult to manufacture polyols of the invention having low residual values for EOC.

| | | | | | | Quantity |
|---|---|---|---|---|---|---|
| | | | Quantity | Quantity | | Hydrogen |
| | | Quantity | Acetic | Dowex | Quantity | Peroxide |
| | | PHSBO | Acid | C-211 | Toluene | Solution |
| Example # | PHSBO | [Parts wt] | [Parts wt] | [Parts wt] | [Parts wt] | [Part wt] |
| 1-1 | PHSBO-60 | 100 | 8.2 | 7.3 | 60 | 31.8 |
| 1-2 | PHSBO-75 | 100 | 10.2 | 9.1 | 60 | 39.8 |
| 1-3 | PHSBO-80 | 100 | 10.9 | 9.7 | 60 | 42.4 |
| 1-4 | PHSBO-83 | 100 | 11.3 | 10.1 | 60 | 44 |
| 1-5 | PHSBO-90 | 100 | 12.3 | 10.9 | 60 | 47.7 |
| 1-6 | PHSBO-100 | 100 | 13.6 | 12.1 | 60 | 53 |
| 1-7 | PHSBO-105 | 100 | 14.3 | 12.7 | 60 | 55.7 |
| 1-8 | PHSBO-110 | 100 | 15 | 13.3 | 60 | 58.3 |

TABLE 2

| | Iodine Value of | | | State |
|---|---|---|---|---|
| Example | PHFESBO | EOC % | Acid value (AV) | of PHFESBO |
| # | gram I₂/100 gram | PHFESBO | of PHFESBO | at 25° C. |
| 1-1 | <4 | 3.5 | <1 mgKOH/g | waxy solid |
| 1-2 | <4 | 4.0 | <1 mgKOH/g | waxy solid |
| 1-3 | <4 | 4.6 | <1 mgKOH/g | soft waxy solid |
| 1-4 | <4 | 4.8 | <1 mgKOH/g | Greasy solid |
| 1-5 | <4 | 4.8 | <1 mgKOH/g | Greasy solid |
| 1-6 | <4 | 5.7 | <1 mgKOH/g | very cloudy liquid |
| 1-7 | <4 | 5.8 | <1 mgKOH/g | almost clear liquid |
| 1-8 | <4 | 6.1 | <1 mgKOH/g | clear liquid |

### 3. Epoxide Ring Opening:

### EXAMPLES 2-1 THROUGH 2-8

Oligomeric polyols are prepared from the epoxidized soybean oil derivatives (PHFESBO) of Examples 1-1 through 1-8. Example 2-1 uses the PHFESBO of Example 1-1 as its starting material, Example 2-2 uses the PHFESBO of Example 1-2 as its starting point and so on. The polyols are made in a 1-Liter, 3-neck round-bottom flask equipped with a mechanical stirrer, thermocouple for contact with the reactants and products, heating mantle, temperature (heat only) controller, a water-cooled condenser, and a nitrogen atmosphere. The flask is charged with 200 to 300 grams of each of the PHFESBO's from Examples 1-1 through 1-8. For each of Examples 2-1 through 2-8, 0.1 wt % of Ring-Opening Catalyst is initially charged to the flask based on the total weight of the PHRESBO present. For Example 2-1 sufficient methanol is added to the flask to provide a molar ratio of 0.62/1.0 hydroxyl to epoxides groups (“OH/EOC”). The higher ratio of hydroxyl groups to epoxides used for Example 2-1 was an attempt to raise the final hydroxyl number of the resulting polyol. For Examples 2-2 through 2-8 sufficient methanol is added to the flask to provide a OH/EOC group molar ratio of 0.33/1.0. The reaction mixture is stirred and the ring opening reaction commences. The temperature increases as the reaction continues. Once the temperature has stabilized, external heat is applied to the flask to raise the temperature as measured by thermocouple to approximately 70° C. The temperature is maintained at 70° C. for one hour.

The polyol product is stripped of excess methanol under a reduced pressure of <5 Torr at 80° C. The resulting polyols have the properties set forth in Table 3.

TABLE 3

| | | | | | Final Iodine | | |
|---|---|---|---|---|---|---|---|
| | PHFESBO | Ratio of | Final OH# | Final AV | Value (gI₂/ | Final | Visc |
| Ex # | Used | OH/EOC | mgKOH/g | mgKOH/g | 100 gram product) | EOC % | Pa · s |
| 2-1 | Ex 1-1 | 0.62/1 | 85 | 1.69 | <4 | 0.4 | 3.68 |
| 2-2 | Ex 1-2 | 0.33/1 | 71 | 2.16 | <4 | 1.08 | NA |
| 2-3 | Ex 1-3 | 0.33/1 | 53 | 0.79 | <4 | 2.3 | 2.3 |
| 2-4 | Ex 1-4 | 0.33/1 | 56 | 0.72 | <4 | 2.52 | 1.64 |
| 2-5 | Ex 1-5 | 0.33/1 | 57 | 1.15 | <4 | 2.31 | 1.88 |
| 2-6 | Ex 1-6 | 0.33/1 | 57 | 0.79 | <4 | 2.85 | 2.79 |
| 2-7 | Ex 1-7 | 0.33/1 | 64 | 0.66 | <4 | 2.83 | 5.15 |
| 2-8 | Ex 1-8 | 0.33/1 | 60 | 0.7 | <4 | 2.99 | 3.75 |

| | Olig | Mono | | | Mw/ | | | Gardener | |
|---|---|---|---|---|---|---|---|---|---|
| Ex # | % | % | Mw | Mn | Mn | Fw | Fn | Color | State |
| 2-1 | 55 | 45 | 2515 | 1626 | 1.55 | 3.81 | 2.46 | 1 | S |
| 2-2 | 68 | 32 | 4586 | 2065 | 2.22 | 5.81 | 2.61 | 1 | L-H |
| 2-3 | 52 | 48 | 2468 | 1488 | 1.67 | 2.34 | 1.4 | 1 | L-H |
| 2-4 | 56 | 44 | 2879 | 1577 | 1.83 | 2.68 | 1.47 | 1 | L-H |
| 2-5 | 58 | 42 | 2975 | 1632 | 1.82 | 3.03 | 1.66 | 1 | L-H |
| 2-6 | 60 | 40 | 3711 | 1707 | 2.17 | 3.77 | 1.75 | 1 | L-H |
| 2-7 | 65 | 35 | 5129 | 1924 | 2.67 | 5.82 | 2.18 | 1 | L-C |
| 2-8 | 62 | 38 | 4194 | 1793 | 2.34 | 4.49 | 1.92 | 1 | L-C |

Referring to Table 3, it can be seen from Example 2-1 that a polyol made from a soybean oil having an initial iodine value of less than 70 grams I₂/100 grams oil results in a polyol that is solid (“S”) at room temperature and therefore will be difficult to include in a typical room temperature polyurethane reactive mixture. It can be further seen that a polyol made from a soybean oil having an initial iodine value of 75 grams I₂/100 grams oil (Ex. 2-2), is a hazy liquid (“L-H”) at room temperature, even though the PHFESBO of Example 1-2 was a solid. When heated to approximately 35-40° C. the polyols of Examples 2-2 through 2-8 become clear liquids (“L-C”) with Gardner Color values of 1.0 or less.

It should be noted that while the final acid value of the polyols of Examples 2-2 and 2-5 are greater than 1.0, the acid number could have been readily lowered by the use of Refined Bleached and Deodorized (RBD) partially hydrogenated soybean oil as the starting material. The acid number could have been further lowered by reducing the peroxide value of the partially-hydrogenated, fully epoxidized vegetable oil derivative prior to the epoxide ring opening reaction. Preferably, the peroxide value is reduced by deodorizing the partially-hydrogenated, fully epoxidized vegetable oil derivative as described above. Further, while the number average molecular weight of the polyol of Example 2-3 is slightly low, it is believed that a polyol made on a larger scale reactor system utilizing the same reactants at the same relative ratios will result in a polyol having a higher molecular weight.

### 4. Production of Comparative Polyol A (“CS-A”):

### a) Epoxidation of 130 IV RBD Soybean Oil

To a 22-Liter 5-neck round-bottom flask equipped with a thermocouple, heating mantle, temperature controller, an internal teflon-coated cooling coil, and a nitrogen sweep are charged 8,000 grams of refined bleached deodorized soybean oil ((130 IV. 40.98 moles C═C) available from Cargill, Incorporated), 1,574 grams glacial acetic acid (26.23 moles), 722 grams of Dowex C-21 I, and 3,400 grams of toluene. The reaction mixture is heated with stirring to 70° C. The heat is turned off and a solution of 30% aqueous hydrogen peroxide is added at ˜31 grams/minute. A total of 5,341 grams of 30% peroxide (47.13 moles) is added over two hours. Cooling water How through the cooling coil is adjusted to maintain a temperature of 70° C.±2° C. To maintain 70° C. cooling is required for the first 4.5 hours of reaction, after which heating is required. The reaction is monitored by measuring the epoxide oxygen content (% EOC) of the toluene diluted product phase. Stirring and cooling are stopped when no further increase in % EOC is observed (˜10 hours).

The aqueous and organic phases are allowed to separate. The Dowex C-211 settles to the bottom with the aqueous phase. The aqueous phase and the Dowex resin are sucked out of the flask (5,850 g. pH 2), and the organic phase is washed successively with ˜3,900 grams of 60° C. water until the water phase has a pH of 7 (typically 5-6 washes).

The washed product is stripped under vacuum to final conditions of <5 Torr at 90° C. Approximately 8,450 grams of epoxidized soybean oil derivative are obtained (97.6% yield, not allowing for sampling and transfer losses.) The epoxidized product has an EOC of 7.00%) and an acid value of 0.55 mg KOH/gram. The epoxidized soybean oil is a clear liquid as produced, but solids may begin to appear after several weeks at room temperature. The epoxidized soybean oil exhibits a Gardner color value of less than 1 when measured at 35° C.

### b) Epoxide Ring Opening by Methanol

An oligomeric polyol is prepared from the epoxidized RBD soybean oil

derivative of step a) above in a 5-Liter, 5-neck round-bottom flask equipped with a two-level agitator, thermocouple, heating mantle, cooling coil, a water-cooled condenser, and a nitrogen atmosphere. The flask is charged with 2,500 grams of epoxidized RBD soybean oil derivative (7.0% EOC, 10.96 moles epoxide) and 103 grams (3.22 moles) of methanol and heated to 55° C. with stirring. Catalyst solution (18.1% of a 48% aqueous HBF₄ in MeOH) is added subsurface through a 316SS tube over 180 minutes. Cooling is required to maintain 55° C. for the first ˜1½ hours of catalyst addition. The EOC of the reaction mixture is measured at one-half hour intervals. Catalyst addition is stopped when the EOC reaches 4.30%. The total HBF₄ over 150 minutes hours is 2.77 grams, or 508 ppm relative to the weight of reactants. The total methanol charge including that in the catalyst is 115 grams (3.61 moles), corresponding to a MeOH/epoxide mole ratio of 0.330.

The partially ring-opened product is stripped to final conditions of <5 Torr at 80° C. The resulting polyol (comparative sample A (“CS-A”)) is a clear yellow liquid product having the properties shown below.

| | |
|---|---|
| Gardner color at 35° C. | <1 |
| Hydroxyl number | 57 mg KOH/gram |
| Epoxide Oxygen | 4.13% |
| Acid Value | 0.48 mg KOH/gram |
| Dynamic Viscosity | 4.21 Pa · s @ 25° C. |
| Mn | 1747 |
| Water | 317 ppm |
| Oligomer content | 58.7% (GPC) |
| Odor, ppm | 20 ppm total volatiles from hexanal, |
| | decanal and nonanal |

### 5. Low Density, Flexible, Yellowing Resistant Foams:

### EXAMPLES 3-2 THROUGH 3-6, 3-8, CX-A, AND CX-B

The polyols of Examples 2-3 through 2-6, 2-8, Comparative Sample A (CS-A),

and a polyol (“CS-B”) made according to the procedure of Example 4 of PCT Publication No. WO 2007/123637 A1, published Nov. 1, 2007, are made into slabstock foams according to the procedure described below. The polyol of Example 2-1 was not made into a foam due to its high melting point and the fact that it is a solid at room temperature. Likewise, the polyol of Example 2-2 was not made into a foam due to the large amount of solids present in the polyol at room temperature.

### Step 1: Procedure for Preparing B Slide

The polyols from Examples 2-2 through 2-6, 2-8, CS-A and CS-B are weighed into a 400 ml plastic beaker that is positioned on an electric scale. Next, the formulation required amount (as delineated in Table 4) of silicone surfactant and amine catalyst are added to the beaker. Next, the formulation required amount of stannous octoate and water (as delineated in Table 4) are added to the batch. The temperate of the B-side is adjusted so that prior to mixing with the polyisocyanate (once you mix the two, the temperature rises rapidly) the combined mixture has a temperature of 19.2° C.±0.3° C. The batch is mixed with an electric, lab duty mixer (Delta ShopMaster brand, Model DP-200, 10 inch shop drill press) equipped with a 2″ diameter mixing blade (ConnBlade Brand, Model 1TC from Conn Mixers Co.) for 23 seconds at 1720 rpm's. Separately, the formulation required amount of TDI (as delineated by Table 4) is weighed out into a 50 ml plastic beaker and is set near the mixing station. The TDI is then added to the polyol mixture and is mixed for 7 seconds. Following this, the mixture is poured into an 83 oz cup and is allowed to free rise. The foam and cup are then placed into a temperature-controlled oven at 100° C. for 15 minutes to cure. At the end of the oven cure, the foam is permitted to cure overnight at room temperature. After curing overnight, the foam is conditioned for 72 hours at 25° C. and 50% relative humidity before testing for physical properties. The physical property test results are reported in Table 5. The physical tests of the foams were carried out under the procedures of ASTM D3574, unless indicated otherwise in the examples.

TABLE 4

| | 40% Incorporation |
|---|---|
| Ingredient | (PPH) |
| Polyol Arcol F-3022 | 60 |
| Oligomeric Polyol | 40 |
| (From Examples 2-1 | |
| through 2.8 CX-A and | |
| CX-B) | |
| Water | 4 |
| TDI | 105 Index* |
| Niax | 1 |
| L-5770 (silicone | |
| surfactant) | |
| Dabco | 0.16 |
| BL-11 (amine | |
| catalyst) | |
| Kosmos | 0.22 |
| K-29 (stannous | |
| Octoate | |

*The amount of TDI used was calculated based on the total water and the hydroxyl number of the polyol to provide an isocyanate index of 105.

TABLE 5
(40% INCORPORATION)

| | | Density | Rebound | 25% IFD | 65% IFD | Support | Tensile |
|---|---|---|---|---|---|---|---|
| Ex # | Polyol | (pcf) | (%) | (N/323 cm²) | (N/323 cm²) | Factor | (kPa) |
| 3-3 | 2-3 | 1.89 | 23 | 7.59 | 18.6 | 2.45 | 84.6 |
| 3-4 | 2-4 | 1.53 | 24 | 21.05 | 36.29 | 1.72 | 65.44 |
| 3-5 | 2-5 | 1.53 | 24 | 25.98 | 41.87 | 1.61 | 97.49 |
| 3-6 | 2-6 | 2.02 | 23 | 8.36 | 17.75 | 2.12 | 63.22 |
| 3-8 | 2-8 | 2.1 | 26 | 8.77 | 20.17 | 2.3 | 58.7 |
| CX-A | CS-A | 1.7 | 26 | 22.11 | 49.22 | 2.23 | 60.74 |
| CX-B | CS-B | 2.08 | 26 | 8.37 | 16.27 | 1.94 | 46.43 |

| | | | | 90% | Yellowness | | |
|---|---|---|---|---|---|---|---|
| | | Elong | Air Flow | Compression | index Initial | YI After | YI After |
| Ex # | Polyol | (%) | (ft³/min.) | Set (% loss) | “YI” E-313 | 21 Days | 18 Weeks |
| 3-3 | 2-3 | 80.87 | 2.25 | 16.98 | 20.77 | 23.04 | 26.13 |
| 3-4 | 2-4 | 63.68 | 3.33 | 15.38 | 20.41 | 22.82 | 25.36 |
| 3-5 | 2-5 | 92.33 | 3.42 | 16.00 | 20.75 | 22.92 | 26.65 |
| 3-6 | 2-6 | 68.24 | 1.33 | 87.69 | 20.53 | 27.35 | 30.52 |
| 3-8 | 2-8 | 63.97 | 0.58 | 17.65 | 19.83 | 38.06 | 40.71 |
| CX-A | CS-A | 94.8 | 2.5 | 16.92 | 20.12 | 38.63 | 44.00 |
| CX-B | CS-B | 61.49 | 0.92 | 19.23 | 20.71 | 38.92 | 42.31 |

As can be seen from Table 5, all the polyols make low density, flexible foams having acceptable mechanical properties.

The foams made from all the polyols are initially white. However, as can be seen from the table, the foams manufactured with the polyol made from PHFESBO of the invention (the polyols of Examples 2-3 through 2-6) retain their white color after being exposed to ambient light for 21 days much better than the foams made from CS-A, CS-B. and the polyol of Ex 2-8 as indicated by their yellowness index (YI). Preferably, the yellowness index is less than 30, more preferably 28 or less, and further more preferably 25 or less after 21 days. In fact, even after 18 weeks, the foams of Examples 2-3 through 2-6 still exhibit a yellowness index of 30.52 or less, compared to the foams made from CS-A, CS-B and the polyol of Ex 2-8, which all have yellowness indexes of at least 40 after 18 weeks of exposure to ambient light. The yellowness indices of the foams are measured by/according to the procedures of ASTM E313.

6. Production of Polyol from a Partially-Hydrogenated, Fully Epoxidized Soybean Oil Derivative:

The purpose of this example is to show the manufacture of a polyol using a PHFESBO and similar size equipment as utilized to manufacture comparative sample A (CS-A)

### a) Epoxidation of 90 Iodine Value (IV) Refined, Bleached, Deodorized (RBD) Hydrogenated Soybean Oil:

A 22-Liter 5-neck round-bottom flask equipped with a thermocouple, heating mantle, temperature controller, an internal teflon-coated cooling coil, and a nitrogen sweep is charged with 8,001 grams of hydrogenated soybean oil (90 IV, 28.37 moles C═C), 1090 grams glacial acetic acid (18.15 moles), 722 grams of Dowex C-211, and 3,446 grams of toluene. The reaction mixture is heated with stirring to 70° C. The heat is turned off and a solution of 30% aqueous hydrogen peroxide is added at ˜31 grams/minute. A total of 3.727 grams of 30% peroxide (32.89 moles) are added over two hours. Cooling water flow through the cooling coil is adjusted to maintain a temperature of 70° C.±2° C. To maintain 70° C., cooling is required for approximately the first 4.5 hours of reaction, after which heating is required. The reaction is monitored by measuring the epoxide oxygen content (% EOC) of the toluene diluted product phase. Stirring and cooling are stopped after 10 hours.

The aqueous and organic phases are allowed to separate. The Dowex C-211 settles to the bottom with the aqueous phase. The aqueous phase and the Dowex resin are sucked out of the flask (4074 g, pH 2), and the organic phase is washed successively with ˜3,900 grams of 60° C. water until the water phase has a pH of 7 (approximately 6 washes).

The washed product is stripped under vacuum to final conditions of <5 Torr at 90° C. A total of 8,258 grams of epoxidized soybean oil derivative are obtained (97.7% yield, not allowing for sampling and transfer losses.) The epoxidized product is a clear liquid as produced, but solids may appear after it cools to room temperature (25° C.), with an EOC of 4.75%) and an acid value of 0.44 mg KOH/gram. The epoxidized soybean oil derivative exhibits a Gardner color value of less than 1 when measured at 35° C.

### b) Epoxide Ring Opening by Methanol

An oligomeric polyol is prepared from the epoxidized hydrogenated soybean oil derivative from step 6 (a) above in a 5-Liter, 5-neck round-bottom flask equipped with a two-level agitator, thermocouple, heating mantle, cooling coil, a water-cooled condenser, and a nitrogen atmosphere. The flask is charged with 2,000 grams of the epoxidized soybean oil derivative epoxide (5.94 moles epoxide) and 62.8 grams of methanol and heated to 55° C. with stirring. Catalyst solution (40% of 48% aqueous HBF₄/60% MeOH) is added subsurface through a 316SS lube at 0.090 grams/min. over 152 minutes. Cooling is required to maintain 55° C. for the first ˜1½ hours of catalyst addition. The EOC of the reaction mixture is measured at one-half intervals. Catalyst addition is stopped when the EOC reaches 2.18%. The total HBF₄ over 152 minutes is 2.63 grams, or 1279 ppm of the reaction mixture. The total methanol charge including that in the catalyst is 71.0 grams (2.22 moles), corresponding to a MeOH/epoxide mole ratio of 0.374.

The partially ring-opened product is stripped to final conditions of <5 Torr at 80° C. The resulting clear liquid product has the properties below.

| | |
|---|---|
| Hydroxyl number | 56.6 mg KOH/gram |
| Epoxide Oxygen | 2.22% |
| Acid Value | 0.69 mg KOH/gram |
| Dynamic Viscosity | 2.9 Pa · s @ 25° C. |
| Water | 48 ppm |
| Oligomer content | 63.7% (GPC) |
| Gardner color at 35° C. | <1 |
| Odor, ppm | 25 ppm total volatiles from hexanal, |
| | decanal and nonanal |

Comparing CS-A and the polyol from this example, it can be seen that the inventive polyol requires about double the amount of HBF₄ catalyst during the ring opening step. While this is a negative characteristic of making the inventive polyol, the unexpected beneficial characteristics of the inventive polyol overcome this limitation/characteristic.

What is claimed is:

1. A method for making a partially hydrogenated, fully-epoxidized vegetable oil derivative suitable for use in manufacturing a polyol for low density, flexible, yellowing resistant polyurethane foam application formulations, the method comprising:
(a) hydrogenating a vegetable oil having an initial iodine value of at least about 120 g I₂/100 gram oil to a final iodine value of from about 70 to 100 g I₂/100 gram oil; and
(b) fully epoxidizing the unsaturated carbon-carbon bounds in the vegetable oil from step (a), to obtain the partially-hydrogenated, fully epoxidized vegetable oil derivative, wherein the vegetable oil derivative exhibits a iodine value of less than 4 g I₂/100 gram vegetable oil derivative, an acid number less than 1 mg KOH, an EOC of from about 4.0 to about 5.7%, and a Gardner Color value of 1 or less.

2. The method of claim 1, wherein the vegetable oil has an initial iodine value of less than about 140 g I₂/100 gram oil.

3. The method of claim 1, wherein the partially hydrogenated fully epoxidized vegetable oil derivative contains 25 ppm or less total volatiles based on hexanal, nonanal and decanal.

4. The method of claim 3, wherein the vegetable oil contains less than 20 ppm total volatiles based on hexanal, nonanal and decanal.

5. The method of claim 1, wherein the method further includes deodorizing the partially-hydrogenated, fully epoxidized vegetable oil derivative from step (b).

6. The method of claim 1, wherein the vegetable oil derivative from step (b) is healed to a temperature of at least 170° C. for a sufficient length of time to reduce the peroxide value of the vegetable oil derivative to less than 10 meq/1000 grams vegetable oil derivative.

7. The method of claim 1, wherein the partially-hydrogenated vegetable oil from step (a) has at least 50% monounsaturated fatty acid groups and less than 40% saturated fatty acid groups.

8. A method for making a partially-hydrogenated, fully epoxidized vegetable oil derivative, the method comprising:
(a) obtaining a partially hydrogenated vegetable oil having an iodine value of 85 to 95 g I₂/100 gram oil; and
(b) fully epoxidizing the unsaturated carbon-carbon bounds in the partially hydrogenated vegetable oil from step (a), to obtain the partially hydrogenated, fully-epoxidized vegetable oil derivative, wherein the vegetable oil derivative exhibits a iodine value of less than 4 g I₂/100 gram vegetable oil derivative, an acid number less than 1 mg KOH/gram, and an EOC of at least 4.5%, a Gardner Color value of 1 or less.

9. The method of claim 8, wherein the partially hydrogenated vegetable oil has at least 65% monounsaturated fatty acid groups and less than 25% saturated fatty acid groups.

10. The method of claim 8, wherein the partially hydrogenated fully epoxidized vegetable oil derivative contains 25 ppm or less total volatiles based on hexanal, nonanal, and decanal.

11. A partially hydrogenated, fully-epoxidized vegetable oil derivative suitable for manufacturing a polyol for use in low density, flexible, yellowing resistant, polyurethane foam applications, the vegetable oil derivative having an iodine value of less than 4 g I₂/100 gram vegetable oil derivative, an acid number less than 1 mg KOH/gram vegetable oil derivative an EOC of from 4.5 to about 5.41%, a Gardner Color value of 1 or less, and 25 ppm or less total volatiles based on hexanal, nonanal, and decanal.

12. The vegetable oil derivative of claim 11, wherein the partially hydrogenated, fully-epoxidized vegetable oil derivative is made by fully epoxidizing a partially-hydrogenated vegetable oil, the partially hydrogenated vegetable oil having an iodine value of 70 to 100 g I₂/100 g vegetable oil, the partially hydrogenated vegetable oil exhibiting a Gardner Color value of 1 or less, and 25 ppm or less total volatiles based on hexanal, nonanal, and decanal.

13. The vegetable oil derivative of claim 12, wherein the partially hydrogenated, fully-epoxidized vegetable oil derivative is made by fully epoxidizing a partially-hydrogenated vegetable oil having an iodine value of 85 to 95 g I₂/100 g vegetable oil.

14. The vegetable oil derivative of claim 13, wherein the partially hydrogenated vegetable oil is made by hydrogenating a vegetable oil having a iodine value of 120 to 135 g I₂/100 gram oil.

15. The partially hydrogenated, fully-epoxidized vegetable oil derivative of claim 11 wherein the vegetable oil has an iodine value of 125 to 140 g I₂/100 gram oil.

16. The partially hydrogenated, fully-epoxidized vegetable oil derivative of claim 12, wherein the vegetable oil is selected from the group consisting of: soybean oil, sunflower oil, corn oil, safflower oil, and mixtures thereof.

17. The partially hydrogenated, fully-epoxidized vegetable oil derivative of claim 12, wherein the vegetable oil comprises soybean oil.

18. The partially hydrogenated, fully-epoxidized vegetable oil derivative of claim 12, wherein the partially hydrogenated vegetable oil has at least 70% monounsaturated fatty acid groups and less than 20% saturated fatty acid groups.

19. The method of claim 1, wherein the partially hydrogenated, fully epoxidized vegetable oil derivative is made by fully epoxidizing a partially hydrogenated vegetable oil having an iodine value of 85 to 95 g I₂/100 g vegetable oil.

20. The method of claim 1, wherein the partially hydrogenated vegetable oil is made by hydrogenating a vegetable oil having an iodine value of 120 to 140 g I₂/100 gram oil.

21. The method of claim 1, wherein the vegetable oil is selected from the group consisting of: soybean oil, sunflower oil, corn oil, safflower oil, and mixtures thereof.

22. The method of claim 1, wherein the vegetable oil comprises soybean oil.

23. The method of claim 8, wherein the method further includes deodorizing the partially-hydrogenated, fully epoxidized vegetable oil derivative from step (b).
